(19) United States
(12) Patent Application Publication
(10) Pub. No.: US 2016/0256117 A1
(43) Pub. Date: Sep. 8, 2016

(54) BLOOD PRESSURE MEASURING METHOD AND APPARATUS

(71) Applicant: SAMSUNG ELECTRONICS CO., LTD., Suwon-si (KR)

(72) Inventors: Chanwook BAIK, Yongin-si (KR); Jisoo KYOUNG, Seoul (KR); Youngzoon YOON, Hwaseong-si (KR)

(73) Assignee: SAMSUNG ELECTRONICS CO., LTD., Suwon-si (KR)

(21) Appl. No.: 14/995,816

(22) Filed: Jan. 14, 2016

(30) Foreign Application Priority Data
Mar. 3, 2015 (KR) 10-2015-0029857

(51) Int. Cl. A61B 5/00; A61B 5/0205

(52) U.S. Cl. CPC A61B 5/7278; A61B 5/7203; A61B 5/02055; A61B 5/6803

(57) ABSTRACT

A method and apparatus for measuring blood pressure are provided. According to one or more exemplary embodiments, the apparatus for measuring blood pressure obtains a blood pressure value by applying a plurality of particular points, sampled at regular intervals from a pulse wave signal detected in an ear area of an object, to a pre-stored blood pressure estimation algorithm.

### CROSS-REFERENCE TO RELATED APPLICATION

This application claims priority from Korean Patent Application No. 10-2015-0029857, filed on Mar. 3, 2015 in the Korean Intellectual Property Office, the disclosure of which is incorporated herein in its entirety by reference.

### BACKGROUND

1. Field

Apparatuses and methods consistent with exemplary embodiments relate to measuring blood pressure.

2. Description of the Related Art

Blood pressure is used as one of a number of measures to determine the health status of an individual and a blood pressure measuring device is frequently used at medical institutes and at home. A cuff-type blood pressure measuring device applies a pressure with a cuff surrounding an area where arterial blood passes, until the blood flow is stopped, and then, measures systolic blood pressure and diastolic blood pressure while slowly reducing the applied pressure. Since pressure is applied when using the cuff-type blood pressure measuring device, a user may feel uncomfortable and carrying the device is inconvenient. Thus, the cuff-type blood pressure measuring device is not adequate for real-time, prolonged monitoring of an individual's continuous change in blood pressure. Accordingly, recently, research into a cuffless-type blood pressure measuring device has made significant progress.

### SUMMARY

One or more exemplary embodiments provide a method and apparatus for obtaining a blood pressure value by applying a plurality of particular points, sampled at regular intervals from a waveform of a pulse wave signal detected in an ear area, to a blood pressure estimation algorithm according to one or more exemplary embodiments.

According to an aspect of an exemplary embodiment, there is provided an apparatus for measuring the blood pressure including: an earphone device including a light emitter configured to emit a light to an ear area of an object, and a light receiver configured to receive at least one of a light transmitted through the object, a light that is radiated from the light emitter and reflected by the object, and a diffused light, and perform photodetection of the at least one light to measure a pulse wave signal; and a signal processor configured to obtain a plurality of particular points, which are sampled at regular intervals from a waveform of the pulse wave signal, and apply the plurality of particular points to a blood pressure estimation algorithm to obtain a blood pressure value.

In addition, the blood pressure estimation algorithm may be obtained through a regression analysis on the plurality of particular points, extracted at regular intervals from the waveform of the pulse wave signal, and the blood pressure value.

In addition, the blood pressure estimation algorithm may be obtained through machine learning for the plurality of particular points, extracted at regular intervals from the waveform of the pulse wave signal, and the blood pressure value.

In addition, the apparatus for measuring the blood pressure may further include an effective signal judging unit judging whether the pulse wave signal is an effective signal.

In addition, the effective signal judging unit may determine the pulse wave signal as the effective signal when a power spectrum value within a predetermined frequency range of the pulse wave signal in a frequency domain is greater than a predetermined value.

In addition, the effective signal judging unit may determine the pulse wave signal as the effective signal when a difference between the highest value and the lowest value of the pulse wave signal in a time domain is greater than a predetermined value.

In addition, the apparatus for measuring the blood pressure may further include a noise filtering unit to eliminate noise components included in the pulse wave signal.

In addition, the blood pressure estimation algorithm may be calibrated by using the pulse wave signal measured from the object and the blood pressure value.

In addition, the apparatus for measuring the blood pressure may further include a movement status determination unit that determines a movement status of the object.

In addition, the blood pressure estimating algorithm may include the plurality of blood pressure estimation algorithms corresponding to the movement status, and the signal processor may obtain the blood pressure value by using the blood pressure estimation algorithm corresponding to the movement status of the object determined by the movement status determination unit, among the plurality of blood pressure estimation algorithms.

In addition, the apparatus for measuring the blood pressure may further include a body temperature detector measuring a body temperature of the object.

In addition, the apparatus for measuring the blood pressure may measure at least one of a heart rate and a degree of oxygen saturation of the object by using the pulse wave signal.

According to an aspect of another exemplary embodiment, there is provided a method of measuring the blood pressure including: receiving a transmitted light from an object receiving a light emitted from the light emitter mounted on an earphone device attached to the object, and at least one of a reflected light and a diffused light from the object receiving the light emitted from the light emitter, and performing a photodetection of lights, and measuring a pulse wave signal; obtaining a plurality of particular points, extracted at regular intervals from a waveform of the pulse wave signal; and applying the plurality of particular points to a pre-stored blood pressure estimation algorithm and obtaining a blood pressure value.

In addition, the method of measuring the blood pressure may further include determining whether the pulse wave signal is an effective signal; in response to the pulse wave signal being determined to be the effective signal, performing the obtaining of the plurality of particular points; and in response to the pulse wave signal being determined not to be the effective signal, receiving at least one of a light transmitted through the object, a light that is radiated from the light emitter and reflected by the object, and a diffused light again to measure the pulse wave signal.

In addition, the method of measuring the blood pressure may further include determining a movement status of the object.

In addition, the blood pressure estimation algorithm may include a plurality of blood pressure estimation algorithms corresponding to the movement status, and the obtaining the blood pressure value may obtain the blood pressure value by using the blood pressure estimation algorithm corresponding to the movement status of the object, among the plurality of blood pressure estimation algorithms.

In one or more exemplary embodiments, a non-transitory computer-readable recording medium having a program recorded thereon to execute methods described above on a computer may be provided.

In one or more exemplary embodiments, a computer program, stored on a medium, to execute methods described above in combination with hardware may be provided.

### BRIEF DESCRIPTION OF THE DRAWINGS

The above and/or other aspects will be more apparent by describing certain exemplary embodiments, with reference to the accompanying drawings, in which:

FIG. 1 is a conceptual diagram illustrating a method of measuring blood pressure;

FIG. 2 is a block diagram illustrating an apparatus for measuring blood pressure according to one or more exemplary embodiments;

FIG. 3 is a block diagram illustrating an apparatus for measuring blood pressure according to another exemplary embodiment;

FIGS. 4A-4F are exemplary diagrams illustrating a process of estimating a blood pressure value by using a pulse wave signal according to one or more exemplary embodiments;

FIGS. 5A-5F are exemplary diagrams illustrating an arrangement of light emitting elements and light receiving elements according to one or more exemplary embodiments;

FIGS. 6A-6E are exemplary diagrams illustrating locations of a photoplethysmograph (PPG) signal detector according to a type of an earphone device;

FIG. 7 is a flow chart describing the method of measuring the blood pressure according to one or more exemplary embodiments; and

FIG. 8 is a flow chart describing the method of measuring the blood pressure according to another exemplary embodiment.

### DETAILED DESCRIPTION

Exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions are not described in detail since they would obscure the description with unnecessary detail.

As used herein, the term “and/or” includes any and all combinations of one or more of the associated listed items. Expressions such as “at least one of,” when preceding a list of elements, modify the entire list of elements and do not modify the individual elements of the list.

It will be understood that although the terms “first”, “second”, etc., may be used herein to describe various components, these components should not be limited by these terms. These terms are only used to distinguish one component from another.

The singular forms “a”, “an” and “the” are intended to include the plural forms as well, unless the context clearly indicates otherwise. It will be further understood that the terms “comprises” and/or “comprising” used herein specify the presence of features, numbers, operations, movements, components, parts, or combinations of these, as described in the specification, but do not preclude the presence or possible addition of one or more other features, numbers, operations, components, parts, or combinations of these.

Throughout the specification, when a portion is “connected” to another portion, it includes not only a case of “directly connected” but also a case of “electrically connected” with another element disposed therebetween. In addition, when a portion “includes” an element, unless the context clearly indicates otherwise, it indicates that other elements may be further included, and other elements are not excluded.

Throughout the specifications, an “earphone device” indicates a device which is plugged into or pressed close to an ear, converts electrical signals to sound signals, transmits vibrations to the ear, and allows a user hear a sound. The earphone device may be of various types such as an in-ear earphone tightly covering an area from the outer ear to the inside of the ear, an open-type earphone placed on earflaps, an ear hook-type earphone, a headphone placed on the head and covering the ears, a headset, a necklace type earphone similar to a Gear Circle earphone and an earphone with clips attachable to earlobes.

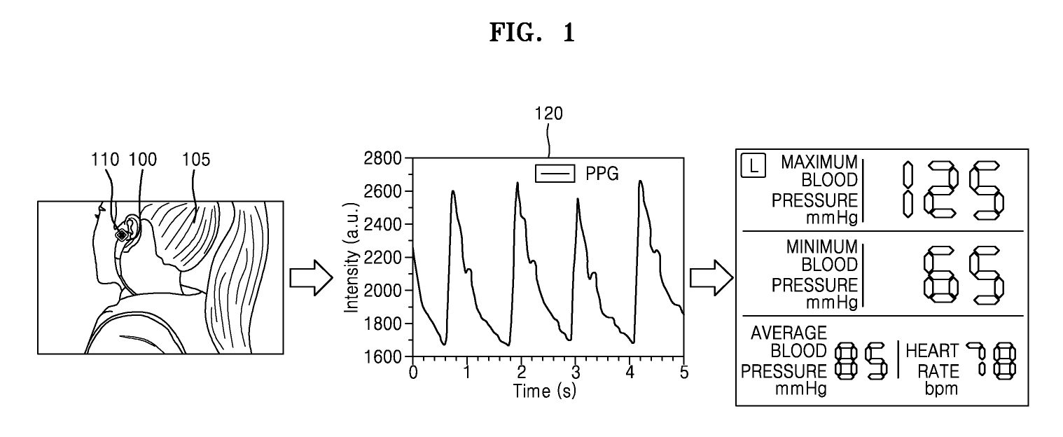

FIG. 1 is a conceptual diagram illustrating a method of measuring blood pressure.

FIG. 1 illustrates that a photoplethysmograph (PPG) signal detector 110 mounted on an earphone device 100 detects a pulse wave using a light signal on an ear area of an object 105 and a blood pressure value is obtained by using a pulse wave signal 120.

A general method of measuring blood pressure by using a cuff includes applying a pressure with the cuff wrapped around an arm, closing and opening an artery, and measuring systolic blood pressure and diastolic blood pressure of a heart by using a sound generated by blood flow. The method of measuring blood pressure by using a cuff may cause discomfort to a user due to the pressure on the arm and inconvenience of carrying the measuring device, and thus, may be inappropriate for prolonged, continuous monitoring.

An apparatus for measuring blood pressure according to one of more exemplary embodiments may detect a biological signal through a sensor mounted on the earphone device, and obtain the blood pressure value using the detected biological signal. Since the biological signal is detected by using a sensor mounted on the earphone device, portability may be enhanced, and prolonged, continuous monitoring may be adequately performed. In addition, since the blood pressure may be simply measured while the user is mobile, self-diagnosis by the user may be simplified, and health management of the user may be supported by transmitting obtained biological signal data to medical professionals.

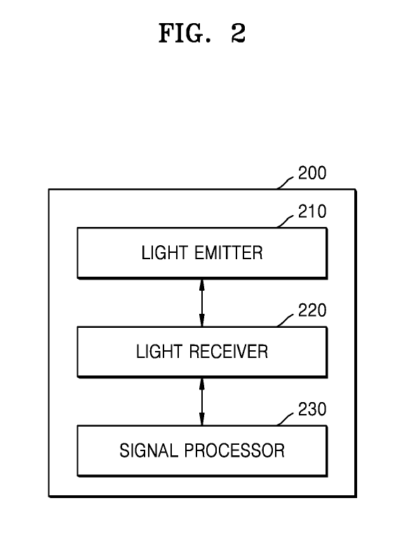

FIG. 2 is a block diagram illustrating an apparatus for measuring blood pressure 200 according to one or more exemplary embodiments.

As illustrated in FIG. 2, an apparatus for measuring blood pressure 200 may include a light emitter 210, a light receiver 220, and a signal processor 230. The apparatus for measuring blood pressure 200 may be realized by more components than those illustrated.

A detailed description of these components is given below.

The light emitter 210 emits light to the object.

The light emitter 210 may be mounted on the earphone device 100. The light emitter 210 may emit the light to an ear area of an object. The ear area includes an ear and an area adjacent to the ear, and may include an external auditory meatus, an earlobe, a tragion portion in front of an auricle, a helix root of the ear, etc.

The light emitter 210 may include at least one light emitting element that emits light to the object. For example, the light emitter 210 may include a light emitting diode (LED), a laser diode (LD), etc.; however, it is not limited hereto.

The light receiver 220 may receive at least one of light that is transmitted through the object 105, light that is radiated from the light emitter 210 and reflected by the object 105, and diffused light, and may perform photodetection of the at least one light to measure the pulse wave signal.

The light receiver 220 may be mounted on the earphone device attached to the object.

The light receiver 220 may include at least one light receiving element that receives at least one light of light transmitted through the object 105, light that is radiated from the light emitter 210 and reflected by the object 105, and diffused light. For example, the light receiver 220 may include a photo diode (PD), a complementary metal-oxide-semiconductor (CMOS) image sensor (CIS), etc.; however, it is understood that one or more other exemplary embodiments are not limited thereto.

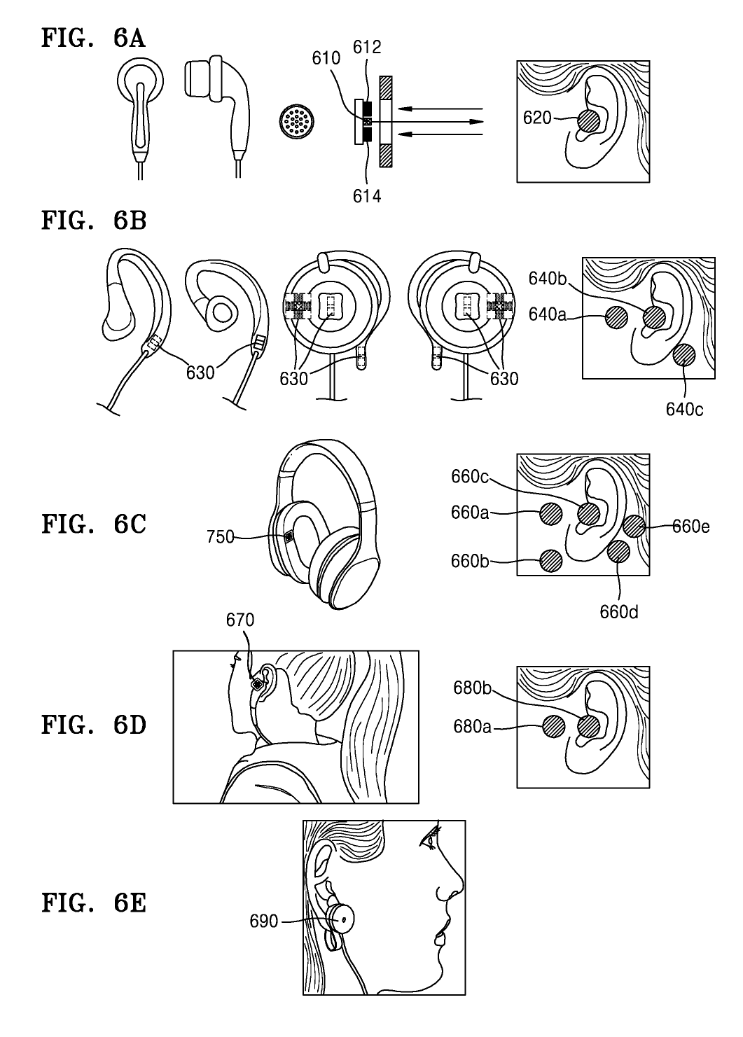

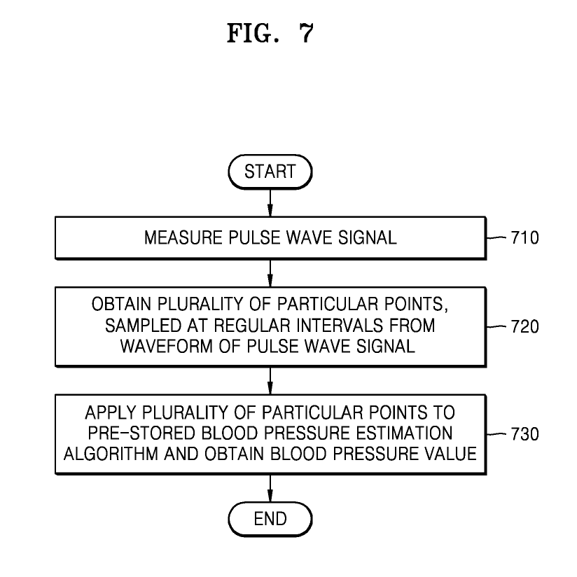

A detailed description of locations and arrangements of the light emitter 210 and the light receiver 220 is given below with reference to FIGS. 6 and 7.

The signal processor 230 may obtain a plurality of particular points sampled at regular intervals from a waveform of the pulse wave signal and apply the plurality of particular points to a pre-stored blood pressure estimation algorithm, thereby obtaining the blood pressure value.

The blood pressure estimation algorithm may be obtained by performing regression analysis on the plurality of particular points, sampled at regular intervals from the waveform of the pulse wave signal, and blood pressure values, according to an exemplary embodiment. For example, the blood pressure estimation algorithm may be obtained by performing linear regression analysis, multiple regression analysis, nonlinear regression analysis, etc. on the plurality of particular points, sampled at regular intervals from the waveform of the pulse wave signal, and blood pressure values. A relation formula may be obtained by assuming that a relation formula between the sampled plurality of particular points and the blood pressure value exists, and analyzing a multitude of data, and the blood pressure estimation algorithm may include such a relation formula.

The blood pressure estimation algorithm may be obtained by machine learning with respect to the plurality of particular points, sampled at regular intervals from the waveform of the pulse wave signal, and blood pressure values, according to another exemplary embodiment. For example, the blood pressure estimation algorithm may be obtained by using an artificial neural network (ANN) algorithm, a k-nearest neighbor (k-NN) algorithm, a Bayesian network algorithm, a support vector network (SVN) algorithm, a recurrent neural network (RNN) algorithm, etc. At least one layer hidden between the sampled plurality of particular points and blood pressure values (the highest blood pressure and the lowest blood pressure) may be set up, and a network may be formed, and a hidden layer matrix may be obtained by repeatedly applying machine learning to such a structure, and using a multitude of data. The blood pressure estimation algorithm may include such a hidden layer matrix.

The blood pressure estimation algorithm may be obtained by using data collected from a randomly sampled population, according to an exemplary embodiment. In other words, the blood pressure estimation algorithm may be obtained by using the data related to the pulse wave signal collected from the randomly sampled population, and blood pressure values.

In one or more exemplary embodiments, the blood pressure estimation algorithm may be obtained by using the data collected from a group classified according to certain characteristics. For example, the blood pressure estimation algorithm may be obtained for each group classified according to physical characteristics, by using the data related to the pulse wave signal collected from each group classified according to physical characteristics such as age, sex, weight and height, and blood pressure. As another example, the blood pressure estimation algorithm may be obtained for each group classified according to status, by using the data related to the pulse wave signal collected from each group classified according to status such as during resting, during exercising, right after exercise, during sleeping, during drinking and during eating, and corresponding blood pressure. As another example, the blood pressure estimation algorithm may be obtained for each group classified according to different times of day, by using the data related to the pulse wave signal collected from each group classified according to each time of day, and blood pressure.

In one or more exemplary embodiments, the blood pressure estimation algorithm may be obtained by using the data collected from a user. For example, the blood pressure estimation algorithm may be obtained by using the data related to the pulse wave signal measured by the user, and blood pressure values. As another example, the blood pressure estimation algorithm may be obtained for each group classified according to each status, by using the data related to the pulse wave signal collected from each group classified according to each status such as during resting, during exercising, right after exercise, during sleeping, during drinking and during eating, and blood pressure. As another example, the blood pressure estimation algorithm may be obtained for each group classified according to different times of day, by using the data related to the pulse wave signal collected from each group classified according to each time of day, and corresponding blood pressure.

The blood pressure estimation algorithm may be obtained from an external device of the apparatus 200 and be stored in the apparatus for measuring blood pressure 200.

The blood pressure estimation algorithm may be calibrated by using the pulse wave signal measured by the object, and the blood pressure value. For example, when the blood pressure estimation algorithm may be obtained by using data collected from a randomly sampled population, the blood pressure estimation algorithm may be calibrated by using the data related to the pulse wave signal measured by the user, and the blood pressure value.

The data related to the blood pressure value to obtain the blood pressure estimation algorithm or to perform a calibration may be obtained by using a general method of measuring blood pressure.

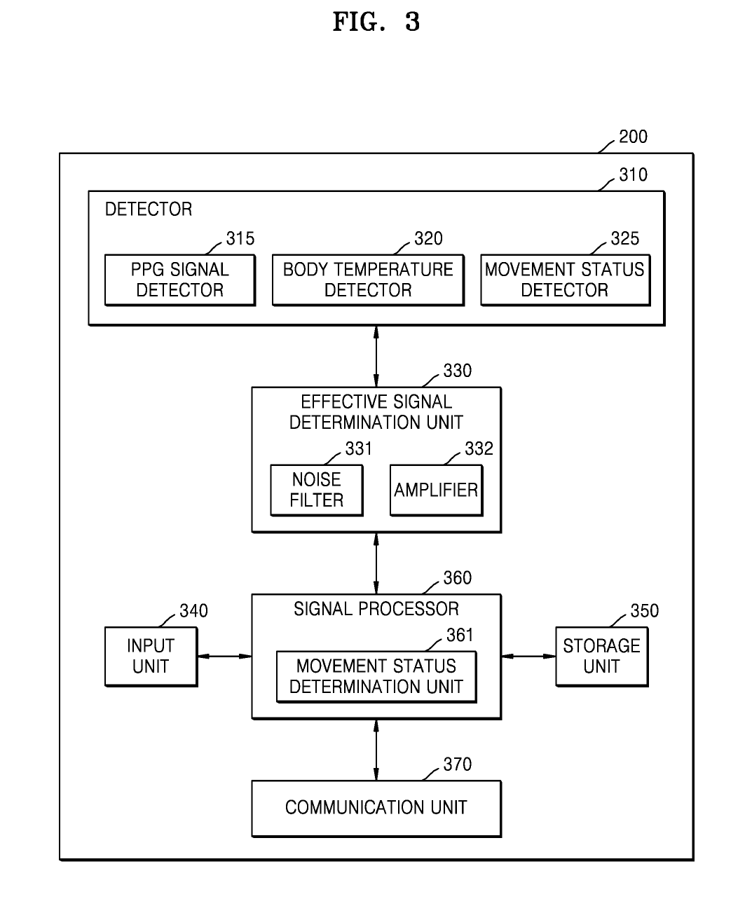

FIG. 3 is a block diagram illustrating an apparatus for measuring blood pressure 200 according to another exemplary embodiment.

As illustrated in FIG. 3, in one or more exemplary embodiments, the apparatus for measuring blood pressure 200 may include a detector 310, an effective signal determination unit 330, an input unit 340, a storage 350, a signal processor 360 and a communication unit 370. However, not all of the illustrated components may be essential components. The apparatus for measuring blood pressure 200 may be realized by more or less components than those illustrated.

In one or more exemplary embodiments, the detector 310 may include a PPG signal detector 315, a body temperature detector 320, and a movement status detector 325.

In one or more exemplary embodiments, the PPG signal detector 315 may be mounted on the earphone device 100 worn by the object, and may detect the pulse wave signal 120 of the object 105. The PPG signal detector 315 may include the light emitter and the light receiver 220. Duplicate descriptions of the light emitter and the light receiver 220 as in FIG. 2 will be omitted.

The apparatus for measuring blood pressure 200 may measure at least one of a heart rate and a degree of oxygen saturation of the object by using the pulse wave signal 120 detected by the PPG signal detector 315.

In one or more exemplary embodiments, the light emitter 210 may emit lights with different wavelengths to the object, and the light receiver 220 may selectively receive each light with a different wavelength.

The degree of oxygen saturation may be obtained by using a difference between an infrared absorption rate at a wavelength with respect to hemoglobin combined with oxygen in blood passing through a capillary and that with respect to hemoglobin not combined with oxygen according to an exemplary embodiment. The light emitter 210 may emit two lights having different wavelengths to an area like the earlobe, and the light receiver 220, located on the opposite side of the light emitter with respect to the earlobe, may receive the light which is transmitted through the earlobe. For example, a light with a wavelength of 660 nm and a light with a wavelength of 940 nm may be used. The oxyhemoglobin, which is hemoglobin combined with oxygen, shows a high absorption rate at 940 nm, and the reduced hemoglobin, which is hemoglobin not combined with oxygen, shows a high absorption rate at 660 nm. The apparatus for measuring blood pressure 200 may calculate the degree of oxygen saturation using the absorption rates of lights having different wavelengths.

The body temperature detector 320 may detect a body temperature of the object according to an exemplary embodiment.

The body temperature detector 320 may detect a body temperature of the object using an infrared sensor. The infrared sensor may be mounted on the earphone device 100 worn by the object 105. The infrared sensor may be adjacent to or in contact with the ear area of the object 105, and receive an infrared light emitted from the ear area of the object 105. The ear area may indicate the ear, an eardrum, and an area including a body portion adjacent to the ear. The body temperature detector 320 may measure a voltage generated according to intensity of the received infrared light. The infrared sensor may include a thermopile to measure the body temperature.

The movement status detector 325 may detect a movement status of the object 105 according to an exemplary embodiment. The movement status detector 325 may be mounted on the earphone device 100 worn by the object 105. The movement status detector 325 may include at least one of an acceleration sensor, a gyro sensor, a multi-axial acceleration sensor, a location information sensor and a global positioning system (GPS) receiver. For example, the movement status detector 325 may detect the movement status such as walking, running, resting and moving speed of the object.

The effective signal determination unit 330 may determine whether the pulse wave signal is an effective signal according to an exemplary embodiment.

For example, the effective signal determination unit 330 may determine the pulse wave signal to be an effective signal when a power spectrum value within a predetermined frequency range of the pulse wave signal in a frequency domain is greater than a predetermined value. A fact is used that components of the pulse wave signal are mainly included in a low frequency range in the frequency domain and noise components are mainly included in a high frequency range in the frequency domain. The predetermined frequency range may be, for example, 0.67 Hz through 3 Hz. As another example, the effective signal determination unit 330 may determine the pulse wave signal as the effective signal when a difference between the highest value and the lowest value of the pulse wave signal in a time domain is greater than a predetermined value.

When the pulse wave signal is determined as the effective signal, the apparatus for measuring blood pressure 200 may estimate at least one of the blood pressure value, the heart rate and the degree of oxygen saturation by using the pulse wave signal. When the pulse wave signal is determined as a non-effective signal, the apparatus for measuring blood pressure 200 may measure the pulse wave signal again through light emitting and light receiving.

The effective signal determination unit 330 may further include a noise filter 331 to eliminate noise components included in the pulse wave signal. In addition, the effective signal determination unit 330 may further include an amplifier 332 to amplify the pulse wave signal to be appropriate for signal processing.

The effective signal determination unit 330 may determine whether a signal detected by the body temperature detector 320 is an effective signal according to another exemplary embodiment. For example, the effective signal determination unit 330 may compare the magnitude of the signal detected by the body temperature detector 320 with a predetermined value, and determine whether the signal is an effective signal based on a result of the comparison.

The effective signal determination unit 330 may determine whether a signal detected in the movement status detector 325 is an effective signal, according to another exemplary embodiment. For example, the effective signal determination unit 330 may compare the magnitude of the signal detected by the movement status detector 325 with a predetermined value and determine whether the signal is an effective signal based on a result of the comparison.

The input unit 340 may receive an input of data to control the apparatus for measuring blood pressure 200 according to an exemplary embodiment.

For example, the input unit 340 may receive an input of physical information such as age, sex, weight and height of the object. As another example, the input unit 340 may receive an input of information related to the status of an object such as a state of resting, exercising, right after exercising, sleeping, drinking, or eating. As another example, the input unit 340 may receive the input of information related to a time when the pulse wave signal is detected.

For example, the input unit 340 may include hardware components such as a key pad, a mouse, a touch pad, a touch screen, a trackball and a jog switch; however, it is not limited thereto.

The storage 350 may store various information processed in the apparatus for measuring blood pressure 200 according to an exemplary embodiment.

For example, the storage 350 may store algorithms or programs used in processing by the apparatus for measuring blood pressure 200, biological signal data input/output and medical data related to information about a physical state of the object. The storage 350 may store data related to the pulse wave signal, body temperatures, movement status, etc. measured by the apparatus for measuring blood pressure 200. The storage 350 may store at least one blood pressure estimation algorithm obtained corresponding to physical characteristics. The storage 350 may store at least one blood pressure estimation algorithm obtained corresponding to a movement status. The storage 350 may store at least one blood pressure estimation algorithm obtained corresponding to a measuring time range. The storage 350 may store at least one blood pressure estimation algorithm obtained corresponding to the user.

For example, the storage 350 may be realized using various kinds of storage media such as a flash memory, a hard disk and electrically erasable programmable read-only memory (EEPROM). In addition, the apparatus for measuring blood pressure 200 may be connected to a web storage or a cloud storage service performing a storing function of the storage 350 on the web.

The signal processor 360 may process the biological signal detected by the detector 310 according to an exemplary embodiment. The signal processor 360 illustrated in FIG. 3 identically corresponds to the signal processor 230 in FIG. 2, and thus, a detailed description thereof will not be repeated here.

The signal processor 360 may calculate at least one of the heart rate, the blood pressure and the degree of oxygen saturation, based on the detected pulse wave signal.

The signal processor 360 may include a movement status determination unit 361 determining the movement status of the object according to an exemplary embodiment. The movement status determination unit 361 may determine the movement status of the object, based on the signal obtained in the movement status detector 325. The movement status determination unit 361 may determine whether the object is in a resting, walking or running state, the moving speed, the moving direction, etc. In addition, the movement status determination unit 361 may calculate an exercise amount of the object, based on a signal obtained in the movement status detector 325. In addition, the movement status determination unit 361 may calculate consumed calories, based on the calculated exercise amount.

For example, when there are a plurality of blood pressure estimation algorithms corresponding to the movement status, the signal processor 360 may obtain the blood pressure value by using the blood pressure estimation algorithm corresponding to the movement status of the object, determined by the movement status determination unit 361, from among the plurality of blood pressure estimation algorithms.

The signal processor 360 may extract one period of a waveform from the biological signal input in a real time. In addition, the signal processor 360 may extract data at regular intervals from one period of a waveform and perform sampling. In addition, the signal processor 360 may convert the biological signal from an analog-type to a digital-type or vice versa.

The communication unit 370 may communicate with external devices or servers, while connected to networks by wire or wirelessly, according to an exemplary embodiment. For example, the communication unit 370 may exchange data with medical hospital servers or other medical apparatuses in hospitals. As another example, the communication unit 370 may transmit measured biological signals and related data to mobile terminals or computers as the user desires.

The communication unit 370 may include more than one component enabling communication with external devices, and for example, may include at least one of a short-range communication module, a wired communication module, and a mobile communication module. The short-range communication module may be a module to perform short-range communication with a device located within a predetermined distance. Examples of short-range communication technology in some exemplary embodiments may include wireless local area network (WLAN), Wi-Fi, Bluetooth, Zigbee, Wi-Fi Direct (WFD), ultra wideband (UWB), infrared data association (IrDA), Bluetooth low energy (BLE), near field communication (NFC), etc.; however, it is not limited hereto. The wired communication module may be a module to perform communication using an electrical signal or an optical signal, and examples of wired communication technology in some exemplary embodiments may include twisted pair cable, coaxial cable, optical fiber cable, Ethernet cable, etc. The mobile communication module may transceive wireless signals with at least one of base stations, external devices, and servers over a mobile communication network. Examples of wireless signals may include a sound call signal, a video call signal or various types of data corresponding to transceiving text/multimedia messages.

The apparatus for measuring blood pressure 200 may include a display according to an exemplary embodiment. The display may display biophysical signals such as a detected pulse wave signal (see FIG. 1), body temperature, and movement status. In addition, the display may display calculated heart rate, blood pressure (see FIG. 1), degree of oxygen saturation, body temperature, exercise amount, consumed calories, etc. The display may display various biological signals obtained from the signal processor 360, in a real time. The display may display various biological signals obtained from the signal processor 360 in a type of time-oriented graph.

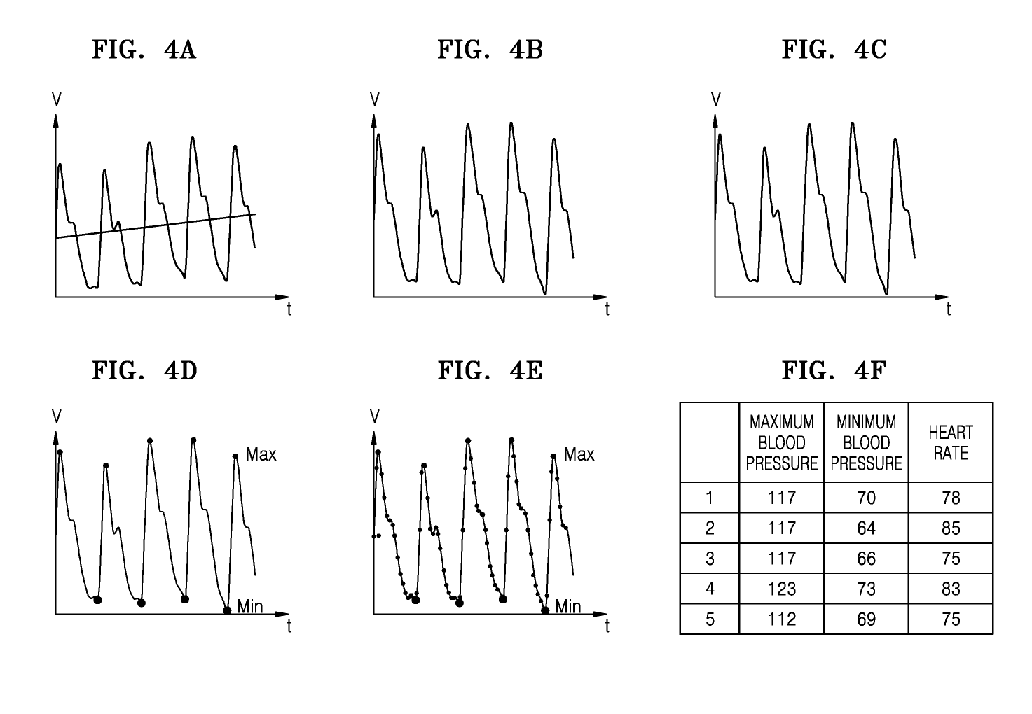

FIG. 4A-4F are exemplary diagrams illustrating a process of estimating a blood pressure value by using a pulse wave signal according to one or more exemplary embodiments.

FIG. 4A illustrates a graph of a measured pulse wave signal and a baseline analysis thereof. The baseline may be a line connecting, for example, middle points between the highest point and the lowest point of each period of waveform of the biological signal. The baseline may be a quadratic function, a cubic function, or a type of function having a plurality of inflection points. FIG. 4A illustrates a type of a linear function having a baseline that increases over time. The apparatus for measuring blood pressure 200 may perform a baseline correction on the obtained pulse wave signal.

FIG. 4B is a graph illustrating a result of the baseline correction.

FIG. 4C is a graph illustrating a result of eliminating noise from the baseline-corrected signal. The apparatus for measuring blood pressure 200 may perform a smoothing function treatment and eliminate high frequency noise components included in the pulse wave signal.

FIG. 4D is a graph illustrating highest points and lowest points for a signal with noise components eliminated. The apparatus for measuring blood pressure 200 may analyze highest points and lowest points of the pulse wave signal and extract a single period sample.

FIG. 4E is a graph illustrating particular points being sampled by a predetermined method. The apparatus for measuring blood pressure 200 may sample particular points at regular intervals. For example, the apparatus for measuring blood pressure 200 may extract particular points at a predetermined time interval for the extracted a single period sample.

FIG. 4F illustrates a result of the blood pressure value obtained by measuring the pulse wave signal every 5 seconds and applying particular points, sampled for one period, to the blood pressure estimation algorithm.

The apparatus for measuring blood pressure 200 may apply a plurality of particular points extracted for every one period of the pulse wave signal to the pre-stored blood pressure estimation algorithm, and obtain the highest blood pressure (systolic blood pressure) and the lowest blood pressure (diastolic blood pressure). In addition, the apparatus for measuring blood pressure 200 may obtain the heart rate using the pulse wave signal.

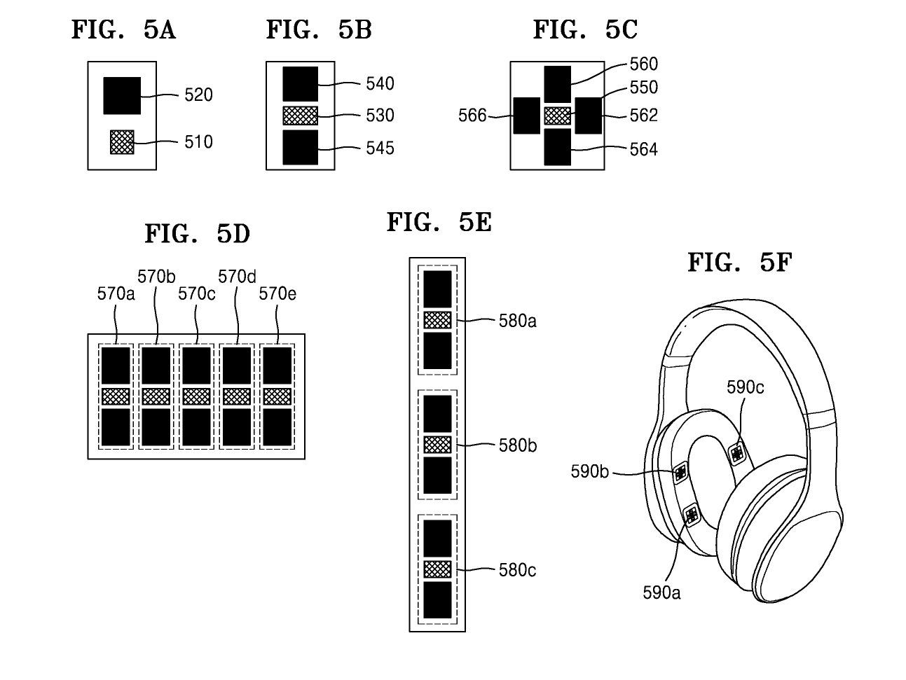

FIG. 5 is an exemplary diagram illustrating an arrangement of light emitting elements and light receiving elements according to one or more exemplary embodiments.

The PPG signal detector 315 may include at least one light emitting element (e.g., light emitter 210) and at least one light receiving element (e.g., light receiver 220).

The PPG signal detector 315 may emit lights having different wavelengths from each of a plurality of light emitting elements according to an exemplary embodiment. Each of a plurality of light receiving elements may receive a light with a particular wavelength corresponding to a wavelength of an emitted light.

The PPG signal detector 315 may include a plurality of channels according to an exemplary embodiment. For example, the apparatus for measuring blood pressure 200 may compare the magnitudes of the pulse wave signal obtained at each channel, and select the pulse wave signal to be used for the blood pressure estimation based on a result of the comparison. As another example, the apparatus for measuring blood pressure 200 may compare a power spectrum value within a predetermined frequency range of the pulse wave signal obtained at each channel, and select the pulse wave signal to be used for the blood pressure estimation based on a result of the comparison.

FIGS. 5A through 5C illustrate the PPG signal detector 315 having a single channel.

FIG. 5A illustrates the PPG signal detector 315 of a type including one light emitting element 510 and one light receiving element 520.

FIG. 5B illustrates the PPG signal detector 315 of a type including one light emitting element 530, and two light receiving elements 540 and 545 arranged with the light emitting element 530 in the middle.

FIG. 5C illustrates the PPG signal detector 315 of a type including one light emitting element 550, and four light receiving elements 560, 562, 564, and 566 with the light emitting element 550 surrounded.

For example, when the PPG signal detector 315 includes a plurality of light receiving elements, the apparatus for measuring blood pressure 200 may obtain the pulse wave signal to be used for the blood pressure estimation after adding all magnitudes of pulse wave signals obtained from each of light receiving elements. In addition, the apparatus for measuring blood pressure 200 may compare the magnitudes of the pulse wave signal obtained at each of light receiving elements, and select the pulse wave signal to be used for the blood pressure estimation based on a result of the comparison. As another example, when the PPG signal detector 315 includes a plurality of light receiving elements, the apparatus for measuring blood pressure 200 may compare a power spectrum value within a predetermined frequency range of the pulse wave signal obtained from each of light receiving elements, and select the pulse wave signal to be used for the blood pressure estimation based on a result of the comparison.

FIGS. 5D and 5E illustrate the PPG signal detector 315 having a plurality of channels.

FIG. 5D illustrates the PPG signal detector 315 of an array type where sets of light emitting elements and light receiving elements 570a, 570b, 570c, 570d, and 570e corresponding to a plurality of channels are horizontally arranged.

FIG. 5E illustrates the PPG signal detector 315 of an array type where sets of light emitting elements and light receiving elements 580a, 580b, and 580c corresponding to a plurality of channels are vertically arranged.

FIG. 5F illustrates the PPG signal detector 315 of a type where sets of light emitting elements and light receiving elements 590a, 590b, and 590c corresponding to a plurality of channels are individually and separately arranged.

For example, when the PPG signal detector 315 includes a plurality of channels, the apparatus for measuring blood pressure 200 may add all magnitudes of pulse wave signals obtained at each channel, and obtain the pulse wave signal to be used for the blood pressure estimation. In addition, the apparatus for measuring blood pressure 200 may compare magnitudes of the pulse wave signal obtained at each channel, and select the pulse wave signal to be used for the blood pressure estimation based on a result of the comparison. As another example, when the PPG signal detector 315 includes a plurality of channels, the apparatus for measuring blood pressure 200 may compare a power spectrum value within a predetermined frequency range of the pulse wave signal obtained at each channel, and select the pulse wave signal to be used for the blood pressure estimation based on a result of the comparison.

FIG. 6 is an exemplary diagram illustrating locations of the PPG signal detector according to a type of an earphone device.

FIG. 6A is an exemplary diagram illustrating the PPG signal detector 315 mounted on an earphone device that is an in-ear type. In general, a speaker used for music appreciation and communication is mounted on an earphone device, and thus, the PPG signal detector 315 needs to be mounted in a manner that does not interfere with the original function of the earphone device. For example, a light emitter 610 and light receivers 612 and 614 may be arranged at the center portion of a speaker that is an annular type. The apparatus for measuring blood pressure 200 may use the pulse wave signal measured at blood vessels of the eardrum or surroundings thereof 620 of the object, and obtain the blood pressure value.

FIG. 6B is an exemplary diagram illustrating the PPG signal detector 630 mounted on an earphone device that is an ear-hook type. The PPG signal detector 630 may be mounted near a speaker or on an earring of the earphone device. Especially, the PPG signal detector 630 may be mounted on the earphone device so as to measure the pulse wave signal at blood vessels of the eardrum or surroundings thereof 640b, in the vicinity of the superficial temporal artery near the tragion 640a, or in the vicinity of the posterior auricular artery of a rear portion of an ear 640c.

FIG. 6C is an exemplary diagram illustrating the PPG signal detector 650 mounted on a headphone device. The PPG signal detector 650 may be mounted near a speaker or an ear-covering area of the headphone device. In particular, the PPG signal detector 650 may be mounted on the headphone device to measure the pulse wave signal at blood vessels of the eardrum or surroundings thereof 660c, in the vicinity of the superficial temporal artery near the tragion 660a and 660b, or in the vicinity of the posterior auricular artery of a rear portion of an ear 660d and 660e.

FIG. 6D is an exemplary diagram illustrating the PPG signal detector 670 mounted on an earphone device that is a Gear-Circle type. The PPG signal detector 670 may be mounted near a speaker or on a supporting stick of the earphone device. In particular, the PPG signal detector 670 may be mounted on the earphone device for measuring the pulse wave signal at blood vessels of the eardrum or surroundings thereof 680b, or in the vicinity of the superficial temporal artery near the tragion 680a.

FIG. 6E is an exemplary diagram illustrating the PPG signal detector 690 mounted on the earphone device that is an earlobe-attachable clip type. In this case, the light emitter of the PPG signal detector 690 is mounted on one side of the clip attached to the earphone device and the light receiver is mounted on the other side of the clip, and the transmitted light through the clip after being emitted from the light emitter may be received.

FIG. 7 is a flow chart describing a method of measuring blood pressure according to an exemplary embodiment.

In operation 710, the apparatus for measuring blood pressure 200 may receive a light transmitted through the object receiving a light from the light emitter mounted on the earphone device, and at least one of a reflected light and a diffused light from the object, perform photodetection, and measure the pulse wave signal.

When the apparatus for measuring blood pressure 200 includes a plurality of light receiving elements, all magnitudes of pulse wave signals obtained from each of the light receiving elements are added to obtain the pulse wave signal to be used for the blood pressure estimation, according to an exemplary embodiment. In addition, the pulse wave signal to be used for the blood pressure estimation may be selected from among pulse wave signals obtained from each of the light receiving elements. For example, the apparatus for measuring blood pressure 200 may compare magnitudes of the pulse wave signal obtained from each of the light receiving element, and select the pulse wave signal to be used for the blood pressure estimation based on a result of the comparison. As another example, the apparatus for measuring blood pressure 200 may compare a power spectrum value within a predetermined frequency range in the pulse wave signal obtained from each of the light receiving element, and select the pulse wave signal to be used for the blood pressure estimation based on a result of the comparison.

When the apparatus for measuring blood pressure 200 includes a plurality of channels, all magnitudes of the pulse wave signal obtained at each channel may be added to obtain the pulse wave signal to be used for the blood pressure estimation. In addition, the pulse wave signal to be used for the blood pressure estimation may be selected from among pulse wave signals obtained at each channel. For example, the apparatus for measuring blood pressure 200 may compare the magnitudes of pulse wave signals obtained at each channel and select the pulse wave signal to be used for the blood pressure estimation based on a result of the comparison. As another example, the apparatus for measuring blood pressure 200 may compare a power spectrum value within a predetermined frequency range of pulse wave signals obtained at each channel and select the pulse wave signal to be used for the blood pressure estimation based on a result of the comparison.

The apparatus for measuring blood pressure 200 may perform a signal preconditioning process to the measured pulse wave signal according to an exemplary embodiment. For example, the signal preconditioning process may include a process of amplifying the pulse wave signal. As another example, the signal preconditioning process may include a process of eliminating noise components included in the pulse wave signal.

In operation 720, the apparatus for measuring blood pressure 200 may obtain a plurality of particular points, sampled at regular intervals from the waveform of the pulse wave signal.

The apparatus for measuring blood pressure 200 may extract a single period sample from the waveform of the pulse wave signal according to an exemplary embodiment. For example, the apparatus for measuring the blood pressure 200 may analyze at least one of the highest point and the lowest point of the waveform of the pulse wave signal, and extract a single period sample. In addition, the apparatus for measuring blood pressure 200 may extract a plurality of particular points at regular intervals for each single period sample. The apparatus for measuring blood pressure 200 may extract a plurality of particular points corresponding to extracted particular points to obtain the blood pressure estimation algorithm.

In operation 730, the apparatus for measuring blood pressure 200 may apply the plurality of particular points to the pre-stored blood pressure estimation algorithm and obtain the blood pressure value.

For example, the blood pressure estimation algorithm may be obtained in advance by performing regression analysis on a plurality of particular points, extracted at regular intervals from the waveform of the pulse wave signal, and blood pressure values, and be stored in the apparatus for measuring blood pressure 200. As another example, the blood pressure estimation algorithm may be obtained in advance by performing regression analysis on a plurality of particular points, extracted at regular intervals from the waveform of the pulse wave signal and blood pressure values, and be stored in the apparatus for measuring blood pressure 200.

The apparatus for measuring blood pressure 200 may use a physical signal obtained from the object, and determine the movement status of the object. In addition, the blood pressure algorithm may include a plurality of blood pressure estimation algorithms corresponding to the movement status. The apparatus for measuring blood pressure 200 may use the blood pressure estimation algorithm corresponding to the movement status of the object, from among a plurality of blood pressure estimation algorithms, and obtain the blood pressure value.

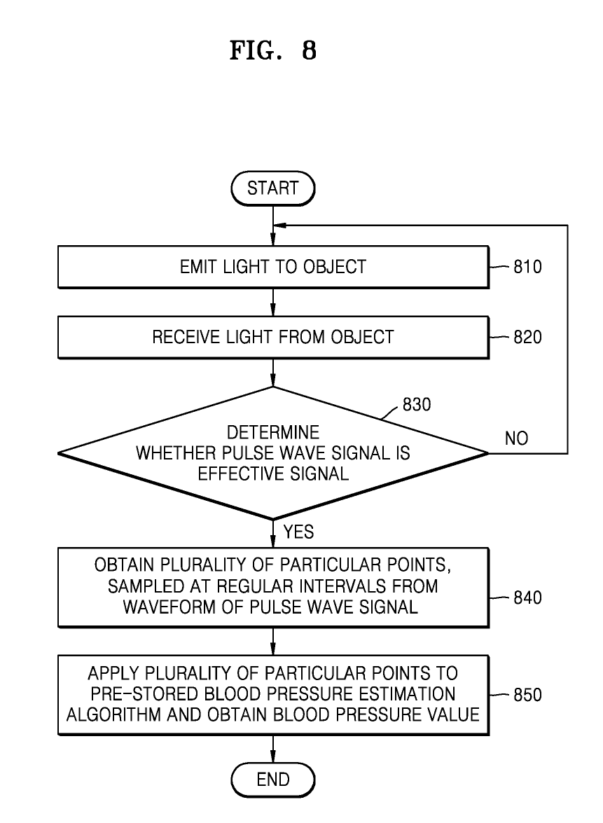

FIG. 8 is a flow chart describing the method of measuring blood pressure according to one or more exemplary embodiments. Operations 840 and 850 in FIG. 8 identically correspond to operations 720 and 730, and thus, detailed descriptions thereof will not be repeated here.

In operation 810, the apparatus for measuring blood pressure 200 may emit a light to the ear area of the object. The ear area indicates an area including the ear and a portion adjacent to the ear, and may include the external auditory meatus, the earlobe, the tragus in front of entrance to the ear canal, a portion under helix root, etc.

In operation 820, the apparatus for measuring blood pressure 200 may receive a light transmitted through the object receiving a light from the light emitter, and at least one of a reflected light and a diffused light from the object, perform photodetection, and measure a pulse wave signal.

In operation 830, the apparatus for measuring blood pressure 200 may determine whether the pulse wave signal is an effective signal.

When the pulse wave signal is determined as the effective signal, operation 840 is performed.

When the pulse wave signal is determined as a non-effective signal, operation 810 is performed, and after performing operations for emitting light and receiving light, the pulse wave signal may be measured again.

Prior to determining whether the pulse wave signal is the effective signal or not, the apparatus for measuring blood pressure 200 may perform a signal preconditioning process on the obtained pulse wave signal, according to an exemplary embodiment. For example, the signal preconditioning process may include a process of amplifying the pulse wave signal. As another example, the signal preconditioning process may include a process of eliminating noise components included in the pulse wave signal.

In operation 840, the apparatus for measuring blood pressure 200 may obtain a plurality of particular points, sampled at regular intervals from the waveform of the pulse wave signal.

In operation 850, the apparatus for measuring blood pressure 200 may apply the plurality of particular points to the pre-stored blood pressure estimation algorithm and obtain a blood pressure value.

The apparatus for measuring blood pressure 200 may enhance convenience in terms of mobility and be utilized as a mobile health management system, by applying the pulse wave signal detected in the ear area to the pre-stored blood pressure estimation algorithm and obtaining the blood pressure value. In addition, since the apparatus for measuring blood pressure 200 obtains the blood pressure value using physical signal sensors mounted on the earphone device, the apparatus for measuring blood pressure 200 may be utilized in wearable devices.

According to one or more exemplary embodiments, the method of measuring blood pressure uses an optical signal and thus, realization using a cuffless-type blood pressure measuring device is feasible.

In addition, since the biological signal may be obtained by the earphone device, realization using a wearable device or a mobile device is feasible.

The apparatus may include a processor, a memory storing and executing program data, a user interface device such as a communication port for communication with external devices, a touch panel, a key and a button, etc., according to one or more exemplary embodiments.

While not restricted thereto, an exemplary embodiment can be embodied as computer-readable code on a computer-readable recording medium. The computer-readable recording medium is any data storage device that can store data that can be thereafter read by a computer system. Examples of the computer-readable recording medium include read-only memory (ROM), random-access memory (RAM), CD-ROMs, magnetic tapes, floppy disks, and optical data storage devices. The computer-readable recording medium can also be distributed over network-coupled computer systems so that the computer-readable code is stored and executed in a distributed fashion. Also, an exemplary embodiment may be written as a computer program transmitted over a computer-readable transmission medium, such as a carrier wave, and received and implemented in general-use or special-purpose digital computers that execute the programs. Moreover, it is understood that in exemplary embodiments, one or more units of the above-described apparatuses and devices can include circuitry, a processor, a microprocessor, etc., and may execute a computer program stored in a computer-readable medium.

The present exemplary embodiments may be illustrated by functional block formations and various processing operations. Such functional blocks may be realized by a multiple number of hardware configurations performing particular functions and/or software configurations. For example, the present exemplary embodiments may adopt integrated circuit (IC) formations such as memory, processors, logic units and look-up tables, which can perform various functions by controlling more than one microprocessor or by other control systems. Similarly to formation elements being capable of being executable by software programming or software factors, the present exemplary embodiments may be realized by programming or scripting languages such as C, C++, Java and assembler, including various algorithms realized by a combination of data structures, processes, routines or other programming formations. Functional aspects may be realized by algorithms executed in more than one processor. In addition, the present exemplary embodiments may adopt related-art technology for electronic environment set-up, signal processing, and/or data processing, etc. Terms such as “mechanism”, “element”, “means” and “formation” may be widely used, and not limited to mechanical and physical formations. Terms above may include meanings of series of routines of software related to a processor, etc.

Particular executions described in the present embodiments are exemplary, and the technical scope thereof should not be limited by any methods. For simplicity of the specification, description of well-understood electronic formations, control systems, software, and other functional aspects of systems above may be omitted. In addition, connections or connecting members between components illustrated in the drawings are exemplary for functional connection and/or physical or circuitry connection, and may be replaceable or realizable as additional and variously functional connections, physical connections or circuitry connections in real apparatuses.

In the present specification, (especially, in the claim section), usage of the definite article “the” and indicative terms similar to it may indicate both singular and plural. In addition, when a range is described to include individual values belonging to the range (unless a description contrary to this exists), it is the same as describing individual values consisting the range in the detailed description. Lastly, unless a description exists to clearly describe a sequence for operations of methods, the operations may be executable in appropriate sequences. Sequences of operations are not limited by descriptions of sequences of the operations. Usage of all examples or exemplary terms (for example, e.g., etc.) is simply to describe technical aspects in detail, and unless limited by the claims, the scope of the present invention is not limited by using examples or exemplary terms. In addition, it will be understood by those of ordinary skill in the art that various corrections, combinations and changes may be made according to design conditions and factors within the scope of the claims or equivalents thereof.

The foregoing exemplary embodiments are merely exemplary and are not to be construed as limiting. The present teaching can be readily applied to other types of apparatuses. Also, the description of exemplary embodiments is intended to be illustrative, and not to limit the scope of the claims, and many alternatives, modifications, and variations will be apparent to those skilled in the art.

What is claimed is:

1. An apparatus for measuring blood pressure, the apparatus comprising:
an earphone device comprising:
a light emitter configured to emit a light to an ear area of an object, and
a light receiver configured to receive at least one of a light transmitted from the ear area, a light that is radiated from the light emitter and reflected by the ear area, and a light diffused from the ear area, and perform photodetection of the at least one light to measure a pulse wave signal; and
a signal processor configured to obtain a plurality of particular points, which are sampled at regular intervals from a waveform of the pulse wave signal, and apply the plurality of particular points to a blood pressure estimation algorithm to obtain a blood pressure value.

2. The apparatus for measuring blood pressure of claim 1, wherein the blood pressure estimation algorithm is obtained through regression analysis of a plurality of particular points, sampled at regular intervals from a waveform of a pulse wave signal, and blood pressure values.

3. The apparatus for measuring blood pressure of claim 1, wherein the blood pressure estimation algorithm is obtained through at least one of an artificial neural network algorithm, a k-nearest neighbor algorithm, a Bayesian network algorithm, a support vector machine algorithm, and a recurrent neural network algorithm, using a plurality of particular points, sampled at regular intervals from a waveform of a pulse wave signal, and blood pressure values.

4. The apparatus for measuring blood pressure of claim 1, wherein the blood pressure estimation algorithm is obtained through machine learning with respect to a plurality of particular points, sampled at regular intervals from a waveform of a pulse wave signal, and blood pressure values.

5. The apparatus for measuring blood pressure of claim 1, further comprising an effective signal determination unit configured to determine whether the pulse wave signal is an effective signal.

6. The apparatus for measuring blood pressure of claim 5, wherein the effective signal determination unit is further configured to determine the pulse wave signal to be the effective signal in response to a power spectrum value within a predetermined frequency range of the pulse wave signal in a frequency domain being equal to or greater than a predetermined value.

7. The apparatus for measuring blood pressure of claim 5, wherein the effective signal determination unit is further configured to determine the pulse wave signal to be the effective signal in response to a difference between the highest value and the lowest value of the pulse wave signal in a time domain being equal to or greater than a predetermined value.

8. The apparatus for measuring blood pressure of claim 1, further comprising a noise filter configured to eliminate noise components of the pulse wave signal.

9. The apparatus for measuring blood pressure of claim 1, wherein the blood pressure estimation algorithm is calibrated by using the pulse wave signal measured from the object and the blood pressure value.

10. The apparatus for measuring blood pressure of claim 1, further comprising a movement status determination unit configured to determine a movement status of the object.

11. The apparatus for measuring blood pressure of claim 10, wherein
the blood pressure estimating algorithm comprises a plurality of blood pressure estimation algorithms corresponding to the movement status, and
the signal processor is further configured to obtain the blood pressure value by using the blood pressure estimation algorithm corresponding to the movement status of the object determined by the movement status determination unit, from among the plurality of blood pressure estimation algorithms.

12. The apparatus for measuring blood pressure of claim 1, further comprising a body temperature detector configured to measure a body temperature of the object.

13. The apparatus for measuring blood pressure of claim 1, wherein at least one of a heart rate and a degree of oxygen saturation of the object is measured by using the pulse wave signal.

14. A method of measuring blood pressure by an optical apparatus, the method comprising:
measuring a pulse wave signal by receiving at least one of a light transmitted from an ear area of an object, a light that is radiated from the light emitter and reflected by the ear area of the object, and a light diffused by the ear area of the object;
performing photodetection of the at least one light;
obtaining a plurality of particular points, sampled at regular intervals from a wave shape of the pulse wave signal; and
applying the plurality of particular points to a blood pressure estimation algorithm to obtain a blood pressure value.

15. The method of measuring blood pressure of claim 14, wherein the blood pressure estimation algorithm is obtained by performing regression analysis on a plurality of particular points, sampled at regular intervals from a waveform of a pulse wave signal, and blood pressure values.

16. The method of measuring blood pressure of claim 14, wherein the blood pressure estimation algorithm is obtained through at least one of an artificial neural network algorithm, a k-nearest neighbor algorithm, a Bayesian network algorithm, a support vector machine algorithm, and a recurrent neural network algorithm, for a plurality of particular points, sampled at regular intervals from a waveform of a pulse wave signal, and blood pressure values.

17. The method of measuring blood pressure of claim 14, wherein the blood pressure estimation algorithm is obtained through machine learning with respect to a plurality of particular points, sampled at regular intervals from a waveform of a pulse wave signal, and blood pressure values.

18. The method of measuring blood pressure of claim 14, further comprising
determining whether the pulse wave signal is an effective signal;
in response to the pulse wave signal being determined to be the effective signal, performing the obtaining of the plurality of particular points; and,
in response to the pulse wave signal being determined not to be the effective signal, receiving at least one of a light transmitted through the object, a light that is radiated from the light emitter and reflected by the object, and a diffused light again to measure the pulse wave signal.

19. The method of measuring blood pressure of claim 14, further comprising determining a movement status of the object.

20. The method of measuring blood pressure of claim 19, wherein the blood pressure estimation algorithm comprises a plurality of blood pressure estimation algorithms corresponding to the movement status, and the obtaining of the blood pressure value comprises obtaining the blood pressure value by using the blood pressure estimation algorithm corresponding to the movement status of the object, from among the plurality of blood pressure estimation algorithms.
